SEQUENCE LISTING (4 sequences)

SEQ ID NO: 1 - DNA, 28 bp, Artificial Sequence
  Primer
aagaattcatgccgtacccgttcaagct

SEQ ID NO: 2 - DNA, 27 bp, Artificial Sequence
  Primer
ctgtcgactcaggctttgttgaagaac

SEQ ID NO: 3 - DNA, 615 bp, Thermus thermophilus
atgccgtacccgttcaagcttcctgacctaggctacccctacgaggccctcgagccccacattgacgccaagaccatggagatccaccaccagaagcaccacggggcctacgtgacgaacctcaacgccgccctggagaagtacccctacctccacggggtggaggtggaggtcctcctgaggcacctcgccgcccttccccaggacatccagaccgccgtgcgcaacaacgggggcgggcacctgaaccacagcctcttctggaggctcctcacccccgggggggccaaggagcccgtgggggagctgaagaaggccattgacgagcagttcgggggcttccaggccctcaaggagaagctcacccaggcggccatgggccggttcggctcgggctgggcctggctcgtgaaggaccccttcggcaagctccacgtcctctccacccccaaccaagacaaccccgtgatggagggcttcacccccatcgtgggcattgacgtctgggagcacgcctactacctcaagtaccagaaccgccgggccgattacctccaggccatctggaacgtcctcaactgggacgtggccgaggagttcttcaataaagcctga

SEQ ID NO: 4 - protein, 204 aa, Thermus thermophilus
MPYPFKLPDLGYPYEALEPHIDAKTMEIHHQKHHGAYVTNLNAALEKYPYLHGVEVEVLLRHLAALPQDIQTAVRNNGGGHLNHSLFWRLLTPGGAKEPVGELKKAIDEQFGGFQALKEKLTQAAMGRFGSGWAWLVKDPFGKLHVLSTPNQDNPVMEGFTPIVGIDVWEHAYYLKYQNRRADYLQAIWNVLNWDVAEEFFKKA

(19) United States
(12) Patent Application Publication
(10) Pub. No.: US 2020/0155652 A1
(43) Pub. Date: May 21, 2020

(54) USE OF MANGANESE SUPEROXIDE DISMUTASE WITH HIGH STABILITY

(71) Applicant: HANGZHOU REDOX PHARMATECH CO., LTD, Jiande (CN)

(72) Inventor: Fanguo MENG, Jiaxing (CN)

(73) Assignee: HANGZHOU REDOX PHARMATECH CO., LTD, Jiande (CN)

(21) Appl. No.: 16/615,140

(22) PCT Filed: Aug. 7, 2018
(86) PCT No.: PCT/CN2018/099148

§ 371 Date: Nov. 20, 2019

(30) Foreign Application Priority Data
Nov. 29, 2017 (CN) 201711228830.X

(51) Int. Cl. A61K 38/44; A61K 9/00; A61K 9/20; A61K 9/48; A61P 3/10; A61P 1/16

(52) U.S. Cl. CPC A61K 38/446; A61K 9/0019; A61P 1/16; A61K 9/48; A61P 3/10; A61K 9/20

(57) ABSTRACT

A method for treating fatty liver, including the step of administering a medicament containing a manganese superoxide dismutase with high stability to a patient. The manganese superoxide dismutase with high stability provided by the present invention can effectively reduce the superoxide level, and has a good therapeutic effect on various types of fatty liver, such as alcoholic fatty liver, non-alcoholic fatty liver, fatty liver accompanied with metabolic syndrome.

### CROSS REFERENCE TO THE RELATED APPLICATIONS

This application is the national phase entry of International Application No. PCT/CN2018/099148, filed on Aug. 7, 2018, which is based upon and claims priority to Chinese Patent Application No. 201711228830.X, filed on Nov. 29, 2017, the entire contents of which are incorporated herein by reference.

### TECHNICAL FIELD

The present invention belongs to the fields of biomedicine, functional food, etc., and particularly relates to use of a superoxide dismutase.

### BACKGROUND

Fatty liver is a widespread chronic disease, which can be divided into alcoholic fatty liver disease (ALD) and non-alcoholic fatty liver disease (NAFLD). Fatty liver is a main cause of liver disease, such as cirrhosis and liver cancer. Non-alcoholic fatty liver disease is characterized by a liver with fat accumulation and metabolic disorders (such as insulin resistance). Hepatic steatosis and metabolic changes caused by long-term high-fat diet or knockout of the obese gene encoding leptin eventually lead to the occurrence of non-alcoholic fatty liver disease, such as liver fibrosis, cirrhosis and liver cancer. Alcoholic fatty liver disease is caused by long-term abuse of alcohol, and can also lead to from a simple steatosis to an end-stage liver disease. The pathogenesis of non-alcoholic fatty liver disease and alcoholic fatty liver disease is very complex, but there are increasing evidences showing that the intestinal barrier injury leads to the translocation of pathogen-associated molecular patterns (pamps) into the extraintestinal tissue, accompanying with the changes of intestinal flora and the occurrence of NAFLD and ALD. Although intestinal barrier injury is known to be an important driver of fatty liver development, there are few effective ways to treat fatty liver symptoms by repairing intestinal barrier injury.

Free radicals have been thought to be involved in the destruction of intestinal barrier for a long time, but few people paid attention to free radicals in the intestinal contents. In vitro studies have shown that intracellular and extracellular superoxide radicals can disrupt the monolayer structure of Caco-2 cells. Currently, extracellular superoxide is thought to be mainly derived from inflammatory cells including macrophages and monocytes. On the other hand, the analysis of metagenomics show that the oxidative stress of the intestinal flora of patients with metabolic diseases was significantly increased. It is worth noting that, in the rat model, the Enterococcus faecalis strain produces extracellular superoxide and produces hydroxyl radicals. Despite the lack of direct evidence showing that the intestinal flora produces superoxide, intestinal microbes may be another source of extracellular superoxide and affect the local environment of intestinal tract. Recent studies have shown that high-fat diet feeding is closely related to the increase of reactive oxygen species (ROS) of intestinal microbes, but the details of the influence of active oxygen of the intestinal flora on the development of fatty liver remain to be studied.

### SUMMARY

The inventors of the present application have investigated the potential effects of intestinal bacterial superoxide on fatty liver and proposed a new approach to treat fatty liver.

Specifically, the present invention provides use of manganese superoxide dismutase with high stability (MS-SOD) for the preparation of a medicament for treating fatty liver, wherein the amino acid sequence of the manganese superoxide dismutase with high stability is set forth in SEQ ID NO: 4.

In a specific embodiment, the fatty liver is alcoholic fatty liver or non-alcoholic fatty liver.

In a specific embodiment, the fatty liver is fatty liver accompanied with metabolic syndrome.

Preferably, the metabolic syndrome is hyperglycemia or insulin resistance.

Preferably, the medicament is administered by oral, injection or intragastric route.

Further preferably, the injection is intravenous injection or intraperitoneal injection.

Preferably, the dosage form of the medicament is a tablet, a capsule, a powder injection, an injection or an aerosol.

Preferably, the medicament further comprises one or more pharmaceutically acceptable excipients, such as diluents, binders, wetting agents, disintegrating agents, solvents and/or buffers, and the like. These excipients are prepared as a medicament in a manner well known in the art with MS-SOD, and the amounts thereof are also known to those skilled in the art.

The manganese superoxide dismutase with high stability provided by the invention can effectively reduce the superoxide level, and has a good therapeutic effect on various types of fatty liver, such as alcoholic fatty liver, non-alcoholic fatty liver, fatty liver accompanied with metabolic syndrome.

### BRIEF DESCRIPTION OF THE DRAWINGS

FIG. 1A shows gene abundance analysis of enzymes associated with oxidative stress;

FIG. 1B shows relative superoxide levels in mouse intestinal feces (n=8-14);

FIG. 2A shows total SOD activity in plasma (n=8);

FIG. 2B shows total SOD activity in intestinal wall (n=8);

FIG. 2C shows total SOD activity in cecal contents (n=7-8);

FIG. 3A shows weight comparison between the high-fat diet group and the MS-SOD treatment group;

FIG. 3B shows relative weights of adipose tissues;

FIG. 3C is a graph of HE and oil red 0 staining for liver (n=6-9);

FIG. 3D is fatty liver scores (n=6-9);

FIG. 3E shows total triglyceride contents in liver (n=7-11);

FIG. 3F shows the mRNA relative expression levels of hepatocyte cytokines and chemokines (n=10-15);

FIG. 3G is the glucose tolerance test (GTT) results (n=10-20);

FIG. 3H is the insulin tolerance test (ITT) results (n=10-20);

FIG. 3I shows fasting blood glucose levels (n=10-20);

FIG. 3J shows fasting insulin levels (n=10-20);

FIG. 3K shows homeostasis model assessment of insulin resistance HOMA-IR index levels (n=10-20);

FIG. 4A shows ethanol concentrations in plasma;

FIG. 4B shows mRNA levels of alcohol dehydrogenase (Adhl) and cytochrome P4502E1 (Cyp2e1) in liver;

FIG. 4C shows ratios of liver to body weight (n=8-10);

FIG. 4D shows alanine aminotransferase levels in plasma (n=8-10);

FIG. 4E shows the results of HE and oil red 0 staining for liver (n=6-10);

FIG. 4F shows the fatty liver scores based on oil red 0 staining (n=6-10);

FIG. 4G shows the total triglyceride contents in liver (n=7-10);

FIG. 4H shows the relative expression levels of Cc12 mRNA in liver (n=8-10);

FIG. 4I shows the relative amounts of superoxide in intestinal feces (n=6-8);

FIG. 4J shows PCoA analysis of cecal bacteria (n=6-8);

FIG. 4K shows fecal albumin levels (n=8-11);

FIG. 4L shows endotoxin levels in plasma (n=8-11);

FIG. 5A shows changes in body weight;

FIG. 5B shows the relative weights of adipose tissues;

FIG. 5C shows the results of HE and oil red 0 staining for liver;

FIG. 5D is fatty liver scores;

FIG. 5E shows the total triglyceride contents in liver;

FIG. 5F shows Cc14 mRNA levels in liver;

FIG. 5G shows the glucose tolerance test (GTT) results;

FIG. 5H shows the insulin tolerance test (ITT) results;

FIG. 5I is a diagram of the variation of the classification of the intestinal flora;

FIG. 5J shows fecal albumin levels; and

FIG. 5K shows endotoxin lipopolysaccharide levels in plasma.

### DETAILED DESCRIPTION OF THE EMBODIMENTS

In the following examples, unless otherwise specified, the detection and monitoring of various indicators were carried out by methods known in the art.

Among them, the kits for the determination of plasma insulin, glucose, total cholesterol and blood ethanol were all purchased from Nanjing JianCheng Bioengineering Institute. The LPS assay kit was purchased from Bioswamp.

The mice used in the examples were all purchased from the Guangdong Experimental Animal Center.

Glucose Tolerance Test (GTT) and Insulin Tolerance Test (ITT): Refer to “Principles and Clinical Applications of Glucose Tolerance Test”, Shanghai Med J. 2009, Vol. 32, No. 5, pp. 440-443.

### Example 1: Preparation of Manganese Superoxide Dismutase with High Stability (MS-SOD)

Using the genome of the Thermus thermophilus HB27 (purchased from the ATCC cell bank of the United States, accession number ATCC BAA-163) as a template, the following primers were used to carry out the amplification: forward primer: 5′-aagaattcatgccgtacccgttcaagct-3′ (SEQ ID NO:1) and reverse primer: 5′-ctgtcgactcaggctttgttgaagaac-3′ (SEQ ID NO: 2), to obtain the target gene; the amplified product was recovered using a recovery kit (purchased from Sangon Biotech (Shanghai) Co., Ltd.), double-digested with enzymes EcoRI and Sal I and ligated into the plasmid vector pET28a(+) (purchased from Sangon Biotech (Shanghai) Co., Ltd.) which was double-digested with the same enzymes. The recombinant plasmid was transformed into competent E. coli BL21 (DE3) (purchased from Sangon Biotech (Shanghai) Co., Ltd.). The strain with the correct MS-SOD nucleotide sequence was screened by sequencing, and the strain was cultured to obtain MS-SOD protein. Wherein, the nucleotide sequence of the gene encoding MS-SOD is as follows:

(SEQ ID NO: 3)
atgccgtacccgttcaagcttcctgacctaggctacccctacgaggccct
cgagccccacattgacgccaagaccatggagatccaccaccagaagcacc
acggggcctacgtgacgaacctcaacgccgccctggagaagtacccctac
ctccacggggtggaggtggaggtcctcctgaggcacctcgccgcccttcc
ccaggacatccagaccgccgtgcgcaacaacgggggcgggcacctgaacc
acagcctcttctggaggctcctcacccccgggggggccaaggagcccgtg
ggggagctgaagaaggccattgacgagcagttcgggggcttccaggccct
caaggagaagctcacccaggcggccatgggccggttcggctcgggctggg
cctggctcgtgaaggaccccttcggcaagctccacgtcctctccaccccc
aaccaagacaaccccgtgatggagggcttcacccccatcgtgggcattga
cgtctgggagcacgcctactacctcaagtaccagaaccgccgggccgatt
acctccaggccatctggaacgtcctcaactgggacgtggccgaggagttc
ttcaataaagcctga.

The amino acid sequence of MS-SOD is as follows:

(SEQ ID NO: 4)
MPYPFKLPDLGYPYEALEPHIDAKTMEIHHQKHHGAYVTNLNAALEKYPY
LHGVEVEVLLRHLAALPQDIQTAVRNNGGGHLNHSLFWRLLTPGGAKEPV
GELKKAIDEQFGGFQALKEKLTQAAMGRFGSGWAWLVKDPFGKLHVLSTP
NQDNPVMEGFTPIVGIDVWEHAYYLKYQNRRADYLQAIWNVLNWDVAEEF
FKKA.

### Example 2: Effect of MS-SOD on Intestinal Flora in Mice Induced by High-Fat Diet

### 1. Establishment of Animal Models:

Male C57BL/6J mice with 6-8 weeks old (purchased from Guangdong Experimental Animal Center) were divided into 3 groups:

(1) Normal control group (NC): fed with low-fat diet (fat-derived calorie 10%) for 18 weeks;
(2) High-fat diet group (HFD): fed with high-fat diet (fat-derived calorie 60%) for 18 weeks;
(3) High-fat diet+MS-SOD group (HFD+MS-SOD): the mice were fed with high-fat diet for 18 weeks, and administered with 3 U MS-SOD/g body weight by intragastric administration from the 8th week. The dose of the intragastric administration was 0.1 ml per mouse, once a day, until the 18th week. The MS-SOD was obtained from the preparation as shown in Example 1. The components of low-fat diet: corn flour, soybean meal, wheat flour, fish, vegetable oil, etc. The components of high-fat diet: lard was added to the low-fat diet.

### 2. Experimental Contents:

(1) Detection of Enzymes Associated with Oxidative Stress

A biotechnology company was entrusted to obtain the gene abundances of manganese superoxide dismutase (Mn-SOD), copper-zinc superoxide dismutase (CuZn-SOD), glutathione peroxidase, catalase and superoxide reductase, by performing metagenomics sequencing on the intestinal flora of NC group and HFD group mice. Specifically:

a. The feces of NC group and HFD group mice were taken separately, 1 g per mouse and then were suspended in 5 ml of 50 mM phosphate buffer (containing 0.5% Tween-20). The resulting samples were repeatedly frozen and thawed at −80° C. and 60° C. for 3 times, to destroy microbial cells.
b. Total RNA of the samples was extracted using Trizol method kit of Invitrogen, and samples were detected by real-time quantitative fluorescent PCR using the ABI 7500 rtPCR system.
c. Determination of the relative expression of the enzyme genes: using 18sRNA as the internal reference gene, the target gene and the internal reference gene were simultaneously amplified by quantitative PCR, and then the relative expression of the target gene was analyzed by Ct comparison method. Primer sequences were obtained from the NIH qPrimerdepot database.

### (2) Detection of Superoxide Levels:

The test was carried out using the superoxide detection kit of Beyotime Biotechnology. The principle of the detection is: superoxide can be detected by using the superoxide to reduce water-soluble tetrazolium-1 (WST-1) to produce soluble colored substances.

a. Adding 200 μl of superoxide buffer, 10 μl of WST-1 solution, and 2 μl of Catalase solution to the detection wells of the ELISA plate;
b. Adding the processed sample to be tested to the detection wells;
c. Incubating for 3 minutes at 37° C.; absorbance was measured at 450 nm.

### 3. Experimental Results

1) High-fat diet feeding increased the superoxide level of intestinal microbes in mice.

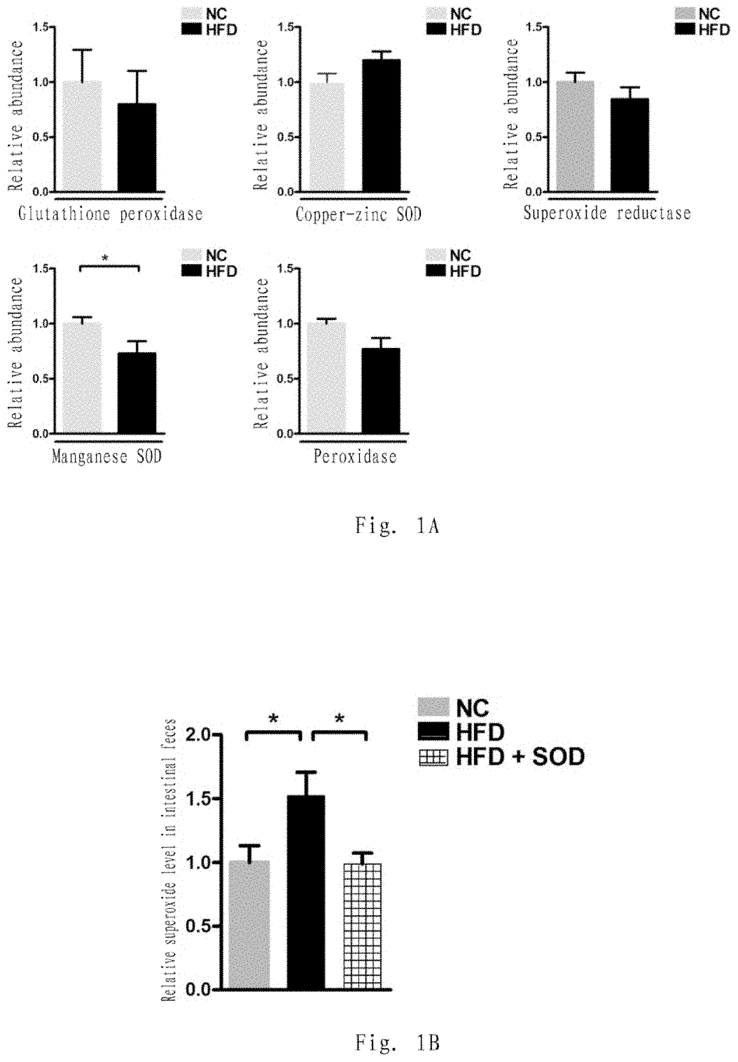

Studies on genes related to reactive oxygen metabolism in mice, including manganese superoxide dismutase, copper-zinc superoxide dismutase, glutathione peroxidase, catalase and superoxide reductase genes were conducted and it was found that except for copper-zinc SOD, all of the gene abundances of other enzymes decreased (FIG. 1A). However, the level of superoxide in the intestinal flora of mice was significantly increased (FIG. 1B). These data clearly indicated that high-fat diet can increase the levels of superoxide in intestinal microbes and reduce the abundances of genes involved in reactive oxygen metabolism.

2) MS-SOD had a repairing effect on the imbalance of superoxide level of intestinal flora induced by high-fat diet.

As above, in order to study the effect of MS-SOD, comparative experiments were conducted at the same time. The results showed that compared with the high-fat diet group mice, the superoxide level in the MS-SOD (which was administered intragastrically) group was reduced to normal level (FIG. 1B), indicating that MS-SOD can repair the imbalance of superoxide level of intestinal flora induced by high-fat diet.

### Example 3: Effect of Intragastric Administration and Injection of MS-SOD on SOD Activity in Intestinal Tract of Mice

### 1. Establishment of Animal Models:

Male C57BL/6J mice with 6-8 weeks old (purchased from Guangdong Experimental Animal Center) were divided into 3 groups:

(1) MS-SOD intragastric administration group: the mice were intragastrically (orally) administered with 6 U MS-SOD/g body weight of per day, and the dose of the intragastric administration was 0.1 ml per mouse, once a day for 7 consecutive days;
(2) Phosphate buffer control group: the mice were administered intragastrically (orally) with a dose of 0.1 ml of phosphate buffer per day, once a day for 7 consecutive days;
(3) MS-SOD intraperitoneal injection group: the mice were intraperitoneally injected with 6 U MS-SOD/g body weight per day, and the dose of the injection was 0.1 ml per mouse, once a day for 7 consecutive days.

### 2. Experimental Contents:

After 7 days, the plasma, jejunum, ileum, colon and cecal contents of each group of experimental mice were taken, and pyrogallol method was used (refer to GB/T5009.171-2003 “Determination of Superoxide Dismutase (SOD) Activity in Health Foods”) to detect the activity of SOD.

### 3. Experimental Results:

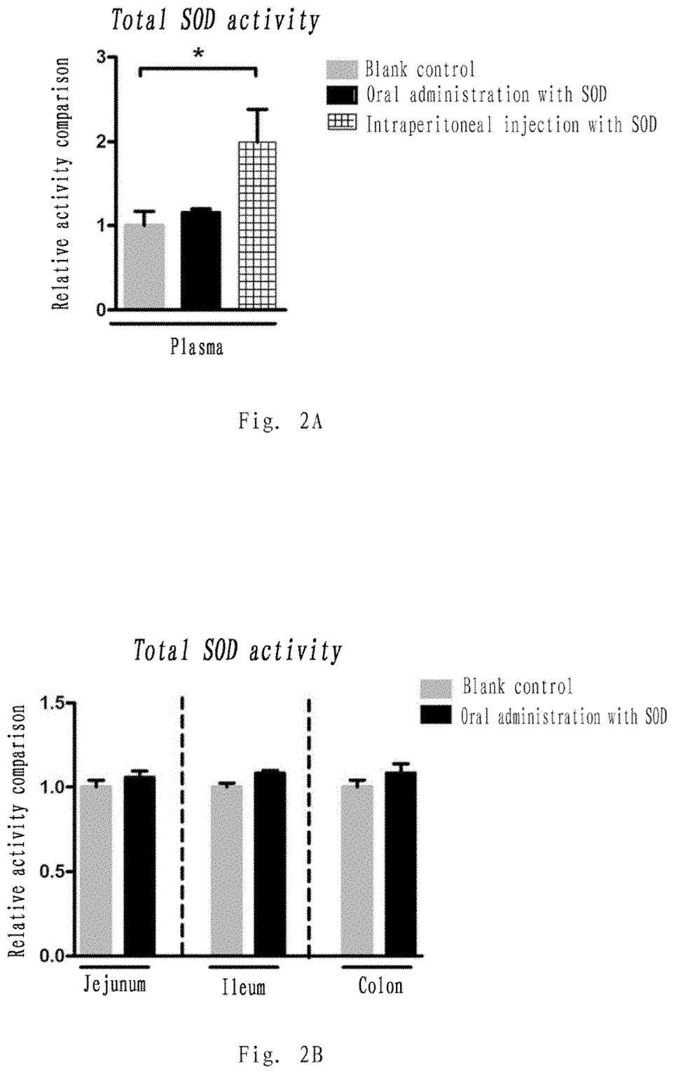

As shown in FIG. 2A, injection of MS-SOD caused an increase of total SOD activity in plasma; in contrast, administering intragastrically (orally) with MS-SOD did not significantly change total SOD in plasma in mice, indicating that oral MS-SOD did not affect extraintestinal organs. As shown in FIG. 2B, the total SOD activity in the jejunum, ileum and colon did not change significantly; however, as shown in FIG. 2C, the SOD activity in the cecal contents was significantly enhanced, indicating that MS-SOD can directly change the oxidation reduction potential in the intestinal bacterial environment.

### Example 4: Effect of MS-SOD on Lipid Accumulation of Liver and Metabolic Syndrome in Mice with High-Fat Diet

### 1. Establishment of Animal Models:

The establishment of animal models was the same as that in Example 2.

### 2. Experimental Results

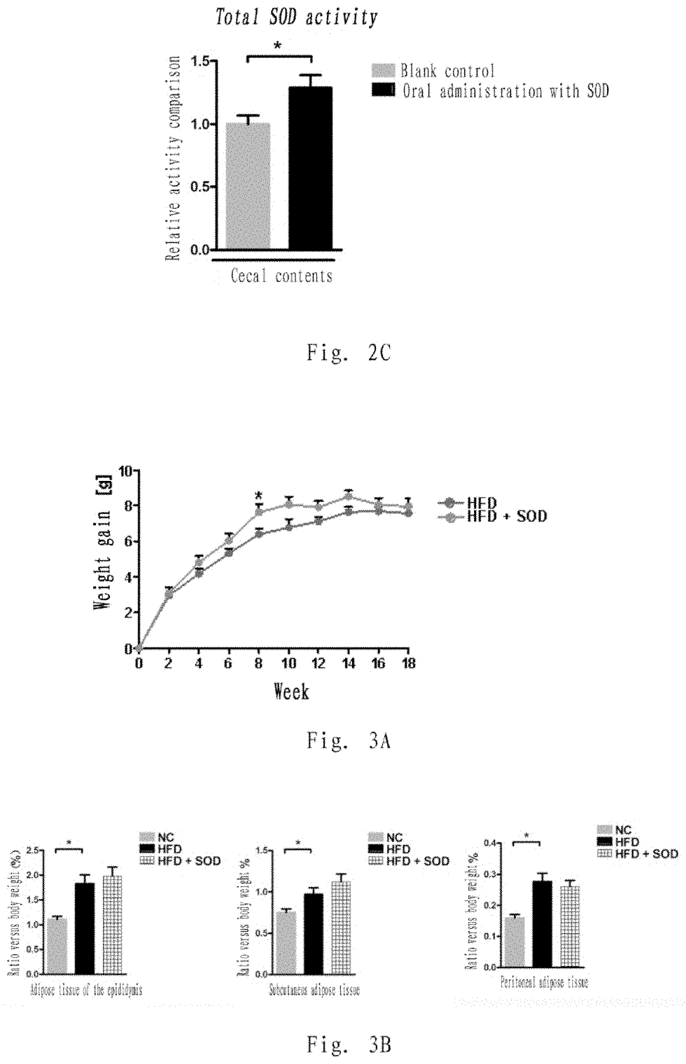

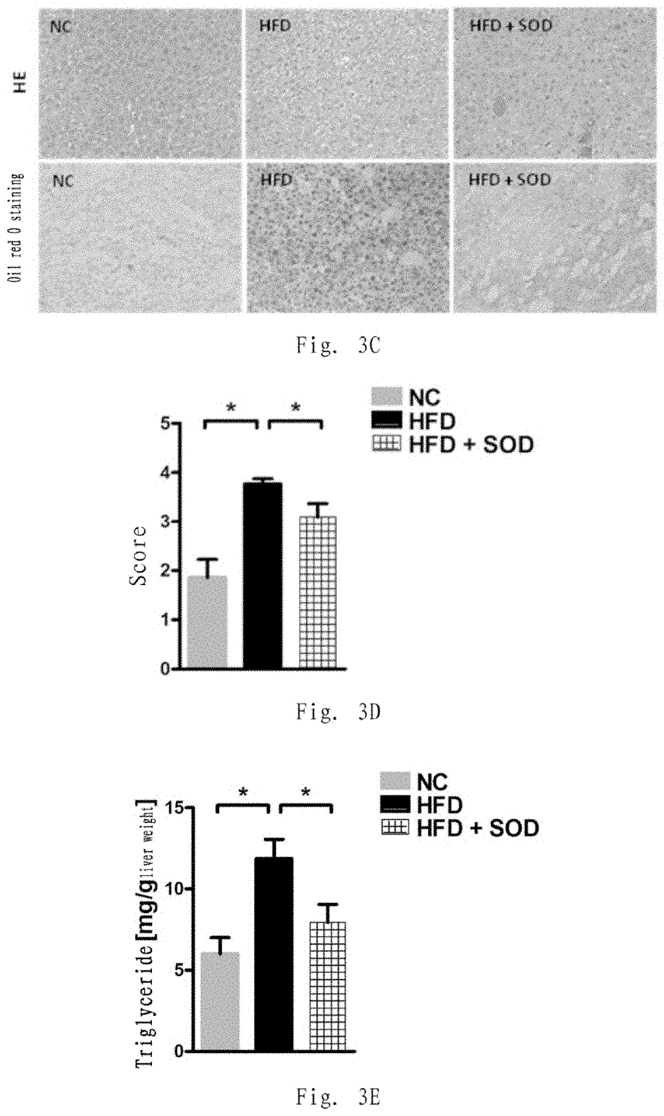

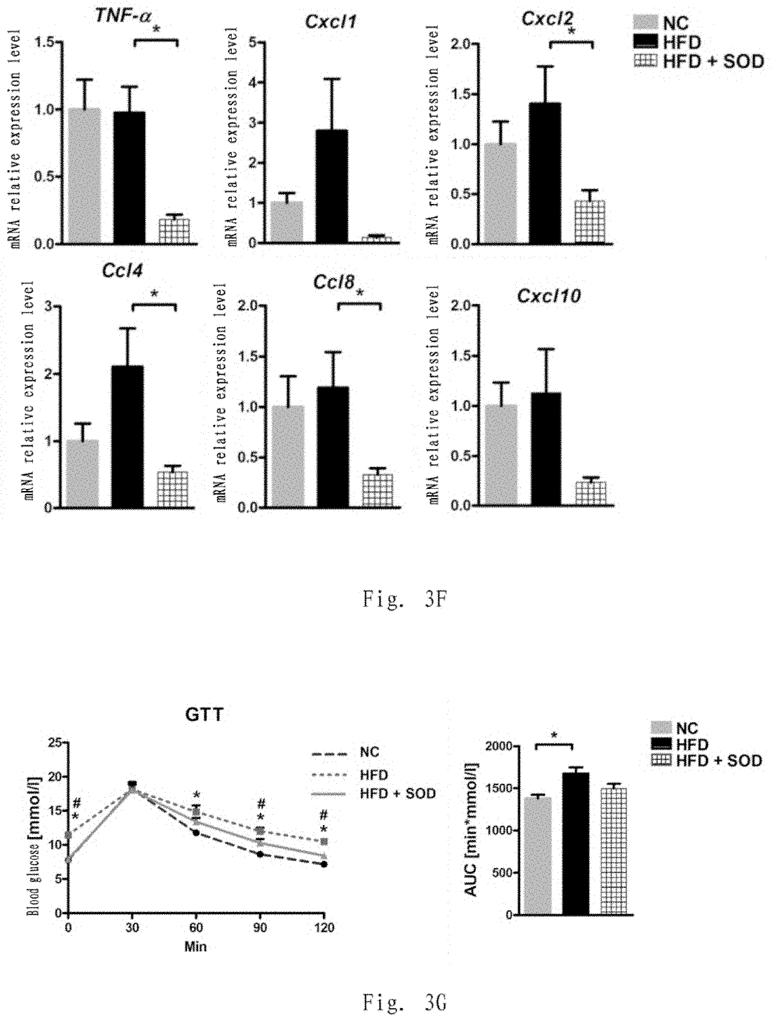

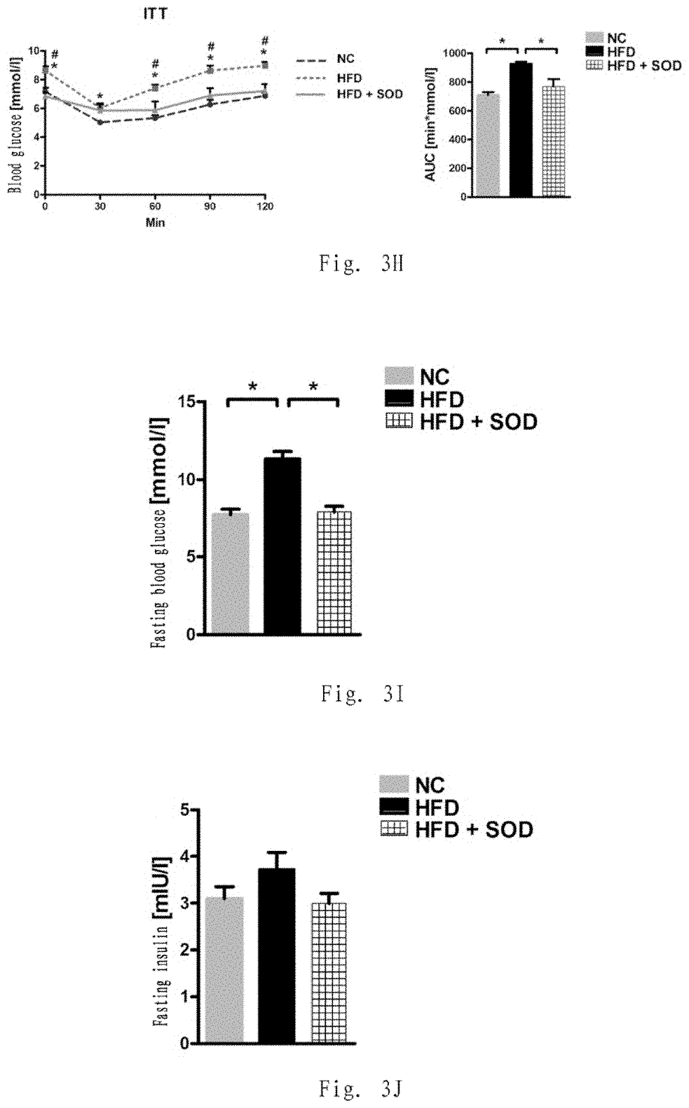

(1) The body weights and the adipose tissue contents of the mice were examined. As shown in FIGS. 3A and 3B, administering intragastrically with MS-SOD did not alter the body weights and adipose tissue contents of the mice. However, it can be seen from the liver section that long-term high-fat diet led to hepatic steatosis. After administering intragastrically with MS-SOD, the fatty liver symptoms were significantly relieved (FIGS. 3C-D), and the triglyceride content of liver was significantly reduced (FIG. 3E).
(2) The mRNA of liver tissue was extracted and reverse transcribed into cDNA. The expression of key cytokines in liver was studied by real-time fluorescent quantitative PCR. The results showed that MS-SOD significantly reduced the mRNA levels of tumor necrosis factor tnf-α, chemokines cc14, cc18, cxc11, cxc12 and cxcl10 during high-fat diet feeding (FIG. 3F).
(3) High-fat diet feeding also led to metabolic disorders, such as glucose tolerance and insulin resistance. Glucose tolerance test (GTT) showed that, compared with the mice fed with high-fat diet, the blood glucose levels were significantly reduced in mice administered intragastrically with MS-SOD (FIG. 3G). Consistent with this, insulin tolerance test results showed that, compared with the mice fed simply with high-fat diet, the mice fed with high-fat diet and administered intragastrically with MS-SOD were more sensitive to insulin (FIG. 3H). Compared with the normal control group, high-fat diet feeding significantly increased fasting blood glucose level and HOMA-IR index, which was a marker of insulin resistance. MS-SOD can almost restore these parameters to normal (FIGS. 3I-K). MS-SOD can improve the impaired insulin sensitivity induced by high-fat diet. It can be seen that MS-SOD can significantly reduce hepatic steatosis and metabolic disorders caused by high-fat diet.

### Example 5: MS-SOD Alleviated the Symptoms of Alcoholic Fatty Liver

### I. Experimental Protocol

### 1. Animal Models:

60 male C57 mice with 8 weeks old (purchased from the Guangdong Experimental Animal Center) were housed in a SPF environment and divided into 3 groups:

Blank control group (control): fed with the Lieber-DeCarli alcohol-free liquid diet for 9 weeks; Model control group: (alcohol, Alc): fed with the Lieber-DeCarli alcoholic liquid diet for 9 weeks;
MS-SOD treatment group (Alc+SOD): fed with the Lieber-DeCarli alcoholic liquid diet for 9 weeks, while administered intragastrically with MS-SOD (3 U/g body weight).

Lieber-DeCarli alcoholic liquid feed (TP4231C, calories including fat 40%, protein 18%, carbohydrate 14%, alcohol 28%, purchased from Trophic Animal Feed High-Tech Co., Ltd, China).

### 2. Experimental Protocol:

The MS-SOD treatment group was administered intragastrically with MS-SOD solution (3 U/g body weight), the amount of intragastric administration was 0.1 ml per mouse, once a day, and the period of intragastric administration was 9 weeks. The blank control group and the model control group were administrated intragastrically with sterile water, and the amount, frequency and period of intragastric administration were the same as those in the treatment group. During the experiment period, fresh feces were collected at 9 am Friday of every two weeks and stored at −80° C. At the 4th, 6th and 8th week of the intragastric intervention, the ALT/AST levels in plasma in mice were monitored by blood collection from eye orbit. On the last day of the experiment, the mice were dissected and tissues were taken and frozen at −80° C.

### 3. Experimental Results:

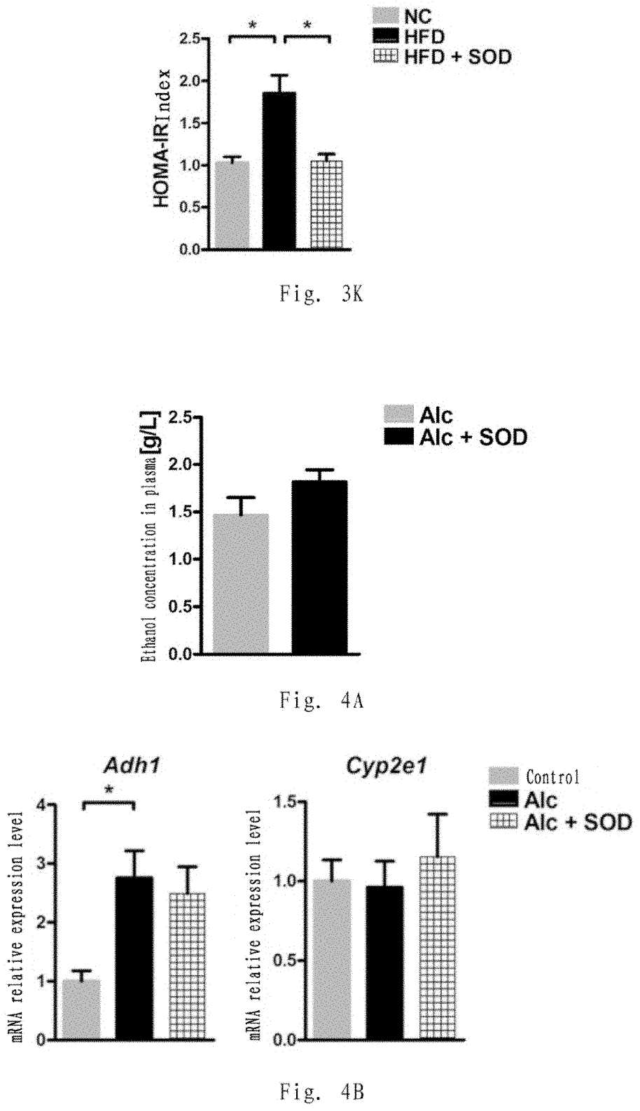

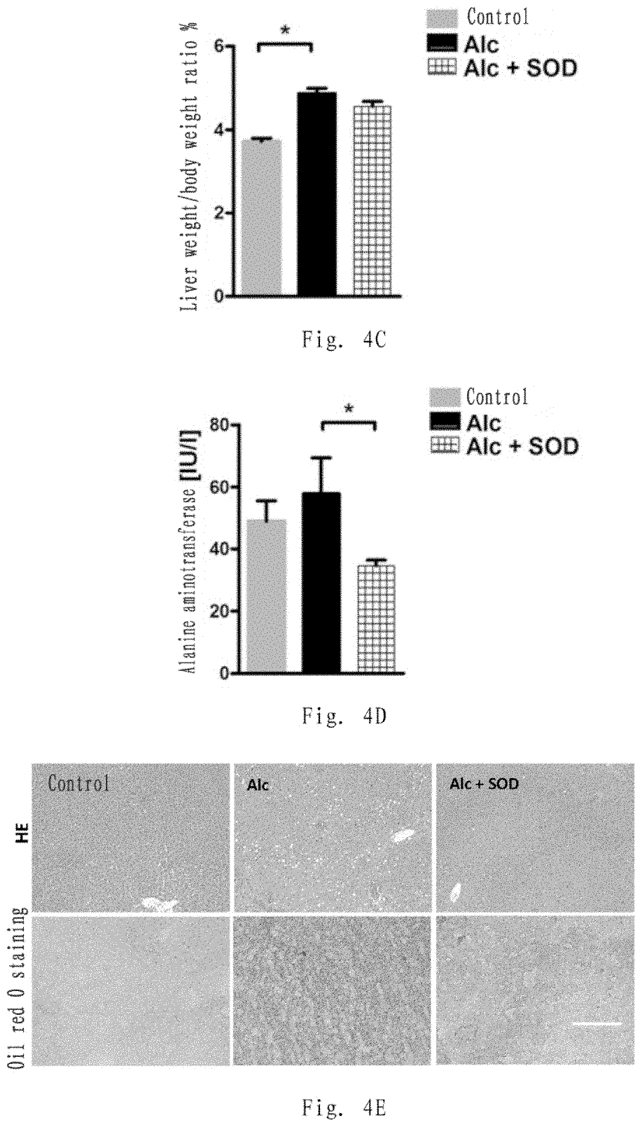

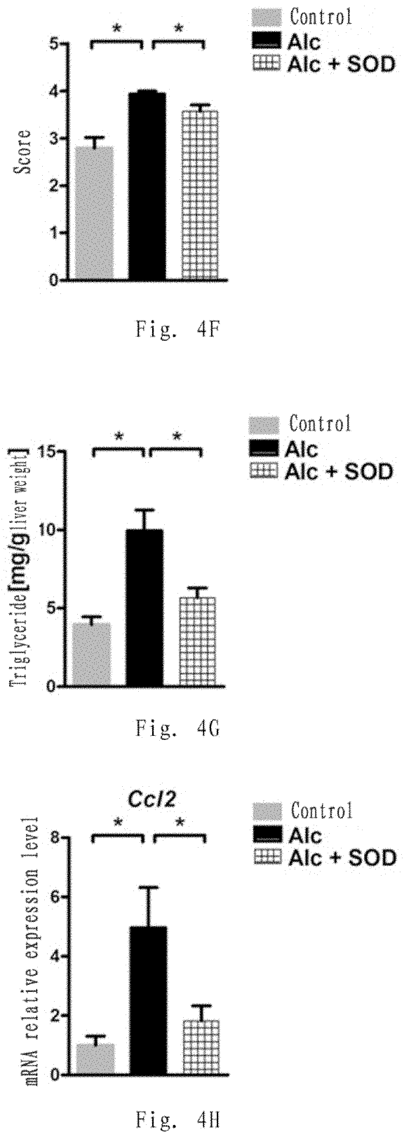

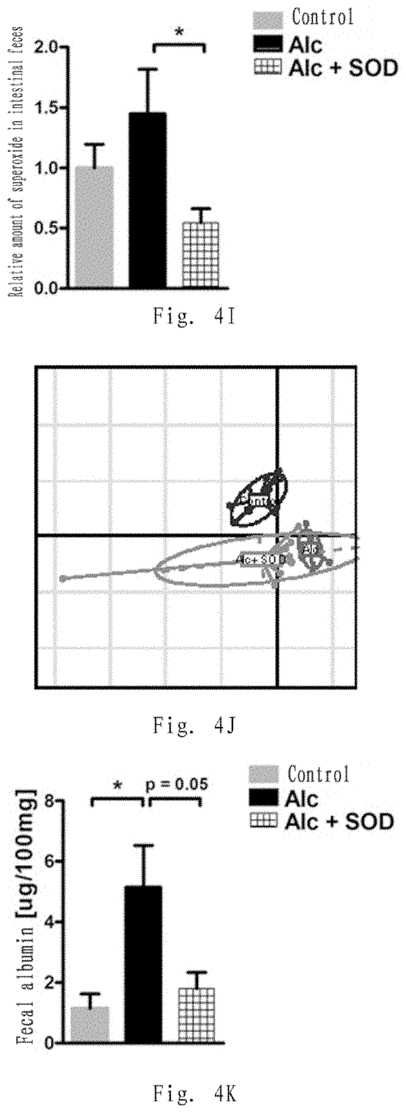

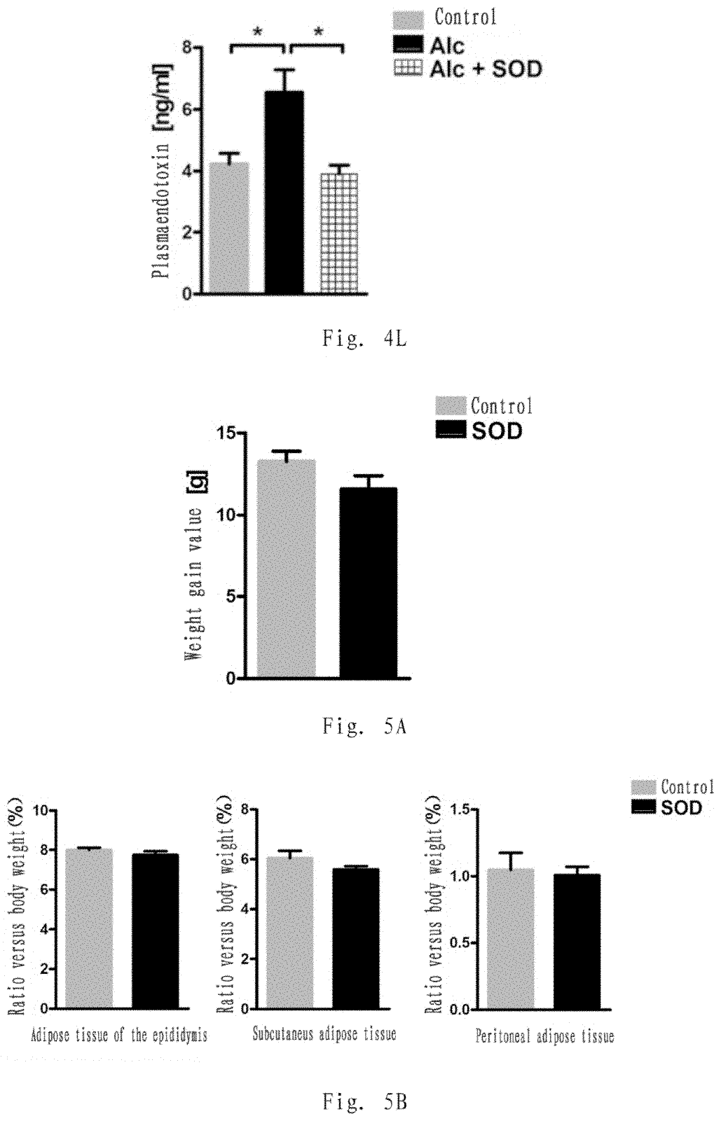

The mice were induced for eight weeks by the liber DeCarli diet. Ethanol metabolism was not affected by MS-SOD, such as ethanol level in plasma and alcohol dehydrogenase, cytochrome p4502E1 expression in liver (FIGS. 4A, 4B). The liver weight/body weight ratio was significantly higher in the alcohol group, and compared with the alcohol group, the liver weight/body weight ratio in the alcohol+SOD group was slightly lower (FIG. 4C). At the same time, alanine aminotransferase level in plasma was significantly reduced in the alcohol+SOD group (FIG. 4D). Importantly, hepatic lipid accumulation and liver cc12 expression were significantly reduced in the MS-SOD group (FIGS. 4E-H). Chronic alcohol abuse showed a clear trend of a higher level of superoxide in the feces, but MS-SOD can significantly reduce superoxide level during alcohol feeding (FIG. 4I). Finally, administering intragastrically with MS-SOD significantly reduced the elevated intestinal permeability and translocation of microbial product caused by chronic alcohol (FIGS. 4J-L). In summary, MS-SOD can improve the symptoms, such as intestinal flora imbalance, intestinal leakage and hepatic steatosis caused by alcohol feeding.

### Example 6: MS-SOD Alleviated Symptoms of Fatty Liver and Metabolic Syndrome in Leptin Gene Deficient Mice (Ob/Ob Mice)

### I. Experimental Protocol

### 1. Animal Models:

Leptin gene deficient mice (ob/ob mice): 6-8 weeks old male mice, purchased from the Guangdong Experimental Animal Center.

### 2. Experimental Protocol

(1) ob/ob mice were fed with MS-SOD (3 U/g body weight) from the 8th week until the 12th week.
(2) Glucose tolerance test (GTT): mice were injected intraperitoneally with glucose (1 g/kg body weight) after 16 hours of fasting. Blood glucose concentrations were measured at 0, 30, 60, 90, and 120 minutes, respectively.
(3) Insulin tolerance test (ITT): mice were injected intraperitoneally with insulin (0.35 U/kg body weight) after 6 hours of fasting. Blood glucose concentrations were measured at 0, 30, 60, 90, and 120 minutes, respectively.

### 3. Experimental Results

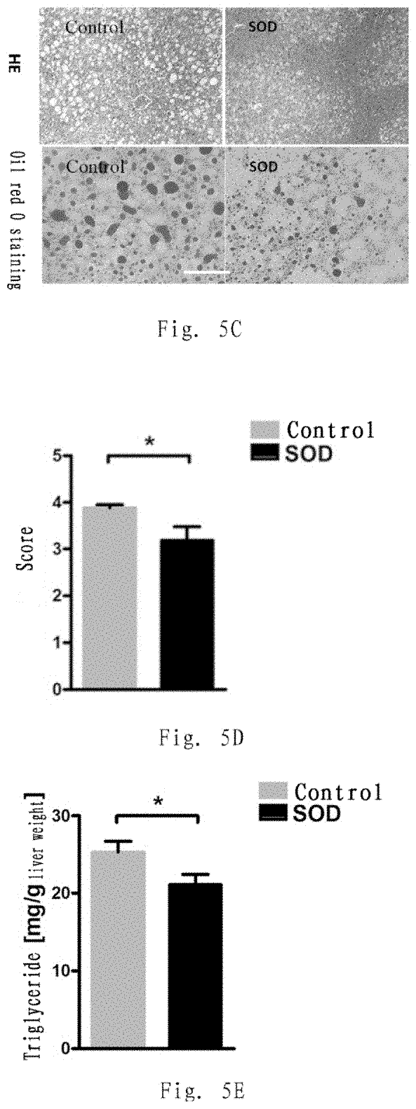

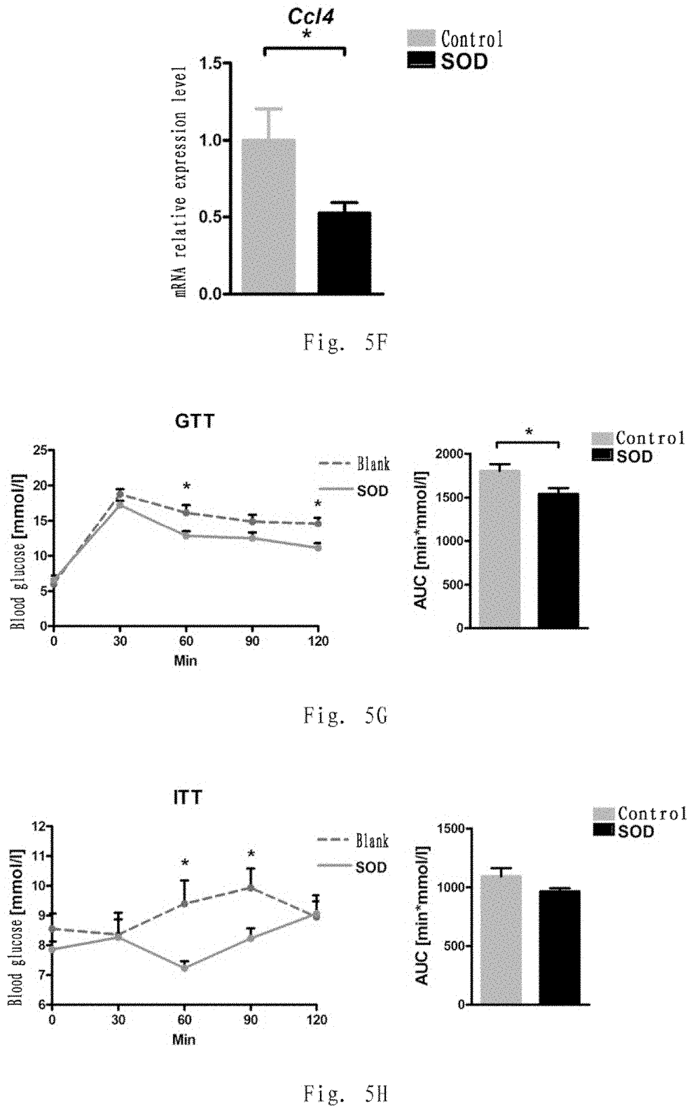

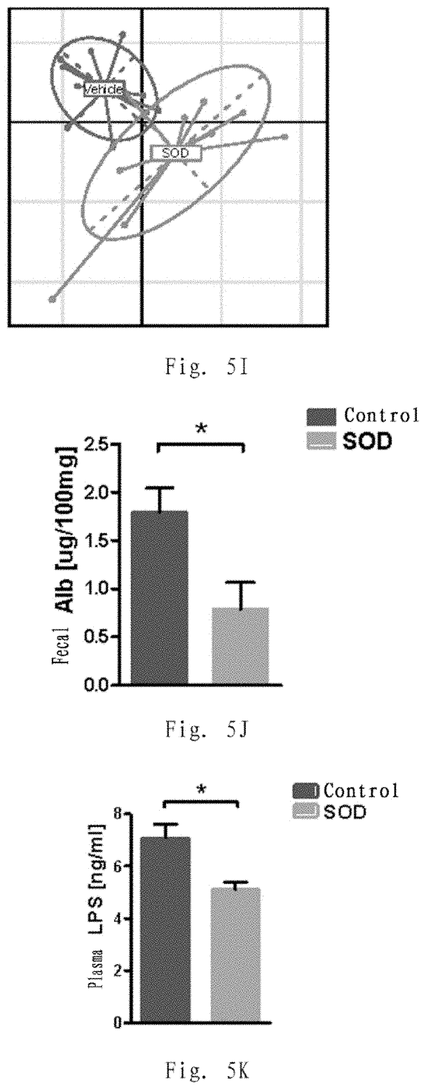

MS-SOD treatment alleviated the fatty liver and metabolic syndrome of leptin gene deficient mice (ob/ob mice). MS-SOD did not significantly change the body weights of ob/ob mice (FIGS. 5A, B), but the adipose tissue index was significantly improved (FIGS. 5C, D), and the triglyceride content of liver was also significantly reduced (FIG. 5E). The mRNA level of liver Cc14 was also significantly reduced (FIG. 5F). GTT and ITT experiments demonstrated that administering intragastrically with MS-SOD resulted in a better control of blood glucose in mice, a significant reduction in blood glucose, and a greater sensitivity to insulin (FIGS. 5G, H). Importantly, similar to the high-fat diet feeding model, MS-SOD treatment significantly changed the composition of the intestinal flora (FIG. 5I). By monitoring the levels of fecal albumin and endotoxin lipopolysaccharide in plasma, it was found that MS-SOD significantly improved intestinal leakage and translocation of bacterial products (FIGS. 5J, K). In conclusion, MS-SOD can relieve the symptoms of fatty liver and metabolic disorders in the OB/OB mouse model.

What is claimed is:

1. A method for treating fatty liver, comprising: a step of administering a medicament containing a manganese superoxide dismutase with high stability to a patient, wherein the amino acid sequence of the manganese superoxide dismutase with high stability is set forth in SEQ ID NO: 4.

2. The method according to claim 1, wherein the fatty liver is alcoholic fatty liver or non-alcoholic fatty liver.

3. The method according to claim 1, wherein the fatty liver is a fatty liver accompanied with metabolic syndrome.

4. The method according to claim 3, wherein the metabolic syndrome is hyperglycemia or insulin resistance.

5. The method according to claim 1, wherein the medicament is administered by oral, injection or intragastric route.

6. The method according to claim 5, wherein the medicament is administered through an injection, and the injection is an intravenous injection or an intraperitoneal injection.

7. The method according to claim 1, wherein a dosage form of the medicament is a tablet, a capsule, a powder injection, an injection or an aerosol.

8. The method according to claim 1, wherein the medicament further comprises one or more pharmaceutically acceptable excipients.

9. The method according to claim 8, wherein the one or more pharmaceutically acceptable excipients are selected from the group consisting of a diluent, a binder, a wetting agent, a disintegrating agent, a solvent and a buffer.

10. The method according to claim 2, wherein the fatty liver is a fatty liver accompanied with metabolic syndrome.

11. The method according to claim 10, wherein the metabolic syndrome is hyperglycemia or insulin resistance.

12. The method according to claim 2, wherein the medicament is administered by oral, injection or intragastric route.

13. The method according to claim 3, wherein the medicament is administered by oral, injection or intragastric route.

14. The method according to claim 4, wherein the medicament is administered by oral, injection or intragastric route.

15. The method according to claim 2, wherein the dosage form of the medicament is a tablet, a capsule, a powder injection, an injection or an aerosol.

16. The method according to claim 3, wherein the dosage form of the medicament is a tablet, a capsule, a powder injection, an injection or an aerosol.

17. The method according to claim 4, wherein the dosage form of the medicament is a tablet, a capsule, a powder injection, an injection or an aerosol.

18. The method according to claim 2, wherein the medicament further comprises one or more pharmaceutically acceptable excipients.

19. The method according to claim 3, wherein the medicament further comprises one or more pharmaceutically acceptable excipients.

20. The method according to claim 4, wherein the medicament further comprises one or more pharmaceutically acceptable excipients.
